(19) United States
(12) Patent Application Publication
(10) Pub. No.: US 2010/0086597 A1
(43) Pub. Date: Apr. 8, 2010

(54) MICROSPHERES FOR THE SUSTAINED RELEASE OF OCTREOTIDE WITH A LOW INITIAL BURST

(75) Inventors: Byung Ho WOO, Broadview Heights, OH (US); Bagavathikanun Chithambara THANOO, Brecksville, OH (US)

Correspondence Address: PEARNE & GORDON LLP, 1801 EAST 9TH STREET, SUITE 1200, CLEVELAND, OH, 44114-3108, US

(73) Assignee: OAKWOOD LABORATORIES LLC, Oakwood Village, OH (US)

(21) Appl. No.: 12/245,915

(22) Filed: Oct. 6, 2008

(51) Int. Cl. A61K 9/10; A61K 38/08

(52) U.S. Cl. 424/486; 514/16

(57) ABSTRACT

This disclosure features microspheres and a method of making them. The microspheres are for sustained release of an octreotide compound with a low initial burst, comprising a poly(D,L-lactide-co-glycolide) polymer matrix and an octreotide compound dispersed in the polymer matrix. The microspheres release less than 1% of a total amount of the octreotide compound within 1 hour at 37° C. and pH 7.4.

### TECHNICAL FIELD

This disclosure relates to the field of polymer-based drug delivery and, in particular, to the delivery of octreotide without an initial burst using polymer microspheres.

### TECHNICAL BACKGROUND

Octreotide is used to treat the symptoms associated with metastatic carcinoid and vasoactive intestinal peptide tumors (VIP-secreting tumors) (Established Clinical Use of Octreotide and Lanreotide in Oncology,” Chemotherapy (2001), 47 (Suppl): 40-53”). Octreotide normalizes the growth hormone levels in acromegaly patients (“Effects of Octreotide Treatment on the Proliferation and Apoptotic Index of GH-Secreting Pituitary Adenomas,” The Journal of Clinical Endocrinology & Metabolism, 86(11): 5194-5200 and “Octreotide Long Acting Release: A Review of its Use in the Management of Acromegaly,” Drugs (2003), 63(22), 2473-2499). Octreotide is indicated for long term maintenance therapy in acromegalic patients for whom medical treatment is appropriate. The goal of treatment in acromegaly is to reduce GH and IGF levels to normal. Octreotide can be used in patients who have had an inadequate response to surgery or in those for whom surgical resection is not an option. It may also be used in patients who have received radiation and have had an inadequate therapeutic response.

Octreotide is a long acting cyclic octapeptide with pharmacologic properties mimicking those of the natural hormone somatostatin. Octreotide is known chemically as L-cysteinamide, D-phenylalanyl-L-cysteinyl-L-phenylalanyl-D-tryptophyl-L-lysyl-L-threonyl-N-[2-hydroxy-1-(hydroxymethyl)propyl]-, cyclic(2→7)-disulfide; [R—(R*,R*)].

A sustained release octreotide formulation is available commercially in the name of Sandostatin LAR. This formulation improves patients comfort; a single monthly injection is used instead of thrice daily subcutaneous (sc) injection. Sandostatin LAR uses a custom polymer, a glucose-PLGA “star” polymer that is specially synthesized.

Biodegradable microsphere delivery formulations are used to release drugs for an extended period of time. The biodegradable microsphere delivery products are often supplied in glass vials or pre-filled syringes filled with powder of drug loaded microspheres and wetting agents. The products are also supplied with a vial or pre-filled syringe which contains a solution of suspending agents, e.g. mannitol, sodium carboxymethylcellulose, polysorbate-80. The products are required to be suspended in the diluent prior to administration. One of the greatest drawbacks of the microsphere delivery products is needle clogging during the withdrawal of suspended microspheres and during administration. The needle clogging may cause an insufficient dose of product and eventually reinjection. Therefore, the products often require relatively large bore gauge needles and a long suspending time to avoid needle clogging. The use of large bore needles causes pain and fear of injection.

The commercially available Sandostatin LAR formulation has only 5% drug content in the microspheres. Approximately 600 mg of microspheres are injected for a 30 mg dose and the injection volume is greater than 2 mL. This might cause excessive pain at the injection site. Additionally, the product requires a large 19 gauge needle for injection into the patient, which might be painful. Thus, there remains a need for compositions and methods for improving the syringeability that minimize needle clogging, pain and fear.

### SUMMARY OF THE INVENTION

In a first embodiment, microspheres for sustained release of an octreotide compound with a low initial burst, comprise a matrix of biodegradable poly(D,L-lactide-co-glycolide) polymer. The polymer does not have any bonded sugar moieties (e.g., glucose) and thus differs from the star polymer. An octreotide compound is dispersed in the polymer matrix. The terms “low initial burst” are defined to mean the microspheres release less than 1% of a total amount of the octreotide compound within 1 hour at 37° C and pH 7.4. The low initial burst can also be characterized by release of less than 1% of a total amount of the octreotide compound within 5 minutes at 37° C and pH of 7.0.

The words, microsphere, microparticle and microcapsule can be used interchangeably with regard to the invention, and mean encapsulation of the octreotide compound by the polymer; the octreotide compound is dispersed in a matrix of the PLGA polymer. In particular, the term microsphere is used throughout this disclosure.

Page 2 of 28

More specifically, the microspheres contain less than 2000 ppm residual solvents. The polymer has a molar ratio of lactide to glycolide ranging from 40:60 to 75:25. The octreotide compound is selected from the group consisting of a free base, an acid addition salt and a complex of octreotide. In particular, the octreotide compound is octreotide acetate.

Also featured is a lyophilized pharmaceutical formulation comprising the microspheres, sodium carboxymethylcellulose and mannitol. In this formulation the octreotide compound is present in an amount of about 3% to about 6% based on a weight of the formulation. The polymer is present in an amount of about 70.0% to about 75.5% by weight of the formulation. The microspheres are present in the formulation in an amount of about 200 mg to about 600 mg. The sodium carboxymethyl cellulose is present in an amount of from about 1.5% to about 5.0% by weight of the formulation. The mannitol is present in an amount of from about 18% to about 21% by weight of the formulation. The formulation is an intramuscular or subcutaneous injectable formulation. The formulation can be reconstituted, for example, with about 2 mL to about 3 mL water for injection. The reconstituted formulation with water for injection can be injectable through a needle that has a size of 20 gauge or smaller (i.e., an inner diameter of 0.584 mm or smaller, in particular, 0.394 mm or even 0.318 mm).

In a second embodiment, a method of making microspheres for extended release of an octreotide compound with a low initial burst, includes preparing a dispersed phase by combining the polymer, the octreotide compound, dichloromethane, methanol and acetic acid. The octreotide compound, polymer, dichloromethane, methanol and acetic acid, can be added in any order or all together. More specifically, the polymer can be dissolved in dichloromethane to form a polymer solution. For example, the concentration of the polymer ranges from about 13% to about 15% of the polymer solution. An octreotide compound can be dissolved in a mixture of acetic acid and methanol to form an octreotide solution. For example, a concentration of the octreotide compound ranges from about 9.2% to about 10.9% of the octreotide solution; and a concentration of the acetic acid ranges from about 5 to 10% and, in particular, from about 5.7% to about 6.7% of the octreotide solution. The polymer solution and the octreotide solution are mixed together to form a dispersed phase. A concentration of the polymer ranges from about 10 to 20% and, in particular, from about 12% to about 15% of the dispersed phase. A concentration of the octreotide compound ranges from about 0.1 to 5% and, in particular, from about 0.8% to about 1.0% of the dispersed phase. A concentration of the acetic acid ranges from about 0.1 to 5% and, in particular, from about 0.4% to about 0.6% of the dispersed phase. Polyvinyl alcohol is dissolved in water to form a continuous phase. The dispersed phase is mixed in the continuous phase to form a microsphere suspension. The dichloromethane, acetic acid, methanol and polyvinyl alcohol are removed from the suspension. Residual dichloromethane and methanol are removed from the microspheres by washing. A diluent is then added to the microspheres comprising sodium carboxymethylcellulose and mannitol. The diluent can be added as a liquid to lyophilized microspheres or can be lyophilized along with the microspheres. When forming a formulation of both lyophilized microspheres and diluent, a concentration of the octreotide compound in the microsphere suspension is adjusted. A suspension of microspheres having the adjusted concentration of the octreotide compound is filled into vials and lyophilized. The vials are stoppered and sealed. A product of the lyophilization is a pharmaceutical formulation for injection.

Another aspect is a process for preparing microspheres for extended release of an octreotide compound with a controlled initial burst. A dispersed phase is prepared by combining poly(D,L-lactide-co-glycolide) polymer, a first solvent for the polymer, the octreotide compound, a second solvent for the octreotide compound and an acid compound. The dispersed phase is mixed in an aqueous continuous phase to form a microsphere suspension. The first solvent, acid compound, and second solvent are removed from the microsphere suspension. Residual first and second solvents are removed from the microspheres by washing. The initial burst of the octreotide compound from the microspheres is measured. The initial burst is raised or lowered to a desired level by adjusting a concentration of at least one of the polymer or the acid compound in the dispersed phase. Then the steps of the method are repeated using the adjusted concentration of polymer or acid compound, or both.

More specifically, the initial burst can be lowered by increasing the concentration of the polymer in the dispersed phase. Also, the initial burst can be lowered by decreasing a concentration of the acetic acid in the dispersed phase. In particular, the first solvent is dichloromethane, the active agent is octreotide acetate, the second solvent is methanol, the acid compound is acetic acid and the aqueous continuous phase includes polyvinyl alcohol.

The octreotide microspheres of this disclosure provide many advantages. They are formed using PLGA polymer, not the custom PLGA-glucose star polymer of the prior art. By tailoring steps of an inventive O/W emulsion process for forming the microspheres to the use of PLGA polymer, the process achieves a unique release profile that has a low initial burst. The inventive microspheres also provide the benefit of being injectable using a smaller needle having a size of 20 gauge or less, which may avoid pain in patients. In addition, the lyophilized octreotide microspheres are quickly resuspended compared to the conventional lyophilized formulation. Many modifications and variations of the invention will be apparent to those of ordinary skill in the art in light of the foregoing disclosure. Therefore, it is to be understood that, within the scope of the appended claims, the invention can be practiced otherwise than has been specifically shown and described.

Many additional features, advantages and a fuller understanding of the invention will be had from the accompanying drawings and the detailed description that follows. It should be understood that the above Summary describes the invention in broad terms while the following Detailed Description describes the invention more narrowly and presents specific embodiments that should not be construed as necessary limitations of the invention as broadly defined in the claims.

### BRIEF DESCRIPTION OF THE DRAWINGS

FIG. 1. Effect of glacial acetic acid concentration in the dispersed phase of microsphere processing on initial release of octreotide PLGA microspheres;

FIG. 2. Effect of polymer concentration in the dispersed phase of microsphere processing on initial release of octreotide PLGA microspheres; and

FIG. 3. Serum octreotide concentration in rats after single administration of octreotide loaded PLGA microspheres (Large Panel). Initial serum octreotide concentration in rats up to 6 hours (Small Panel).

### DETAILED DESCRIPTION OF THE INVENTION

This disclosure provides a pharmaceutical formulation for the sustained release of an octreotide compound (e.g., octreotide acetate) either in vivo or in vitro with a low initial burst. The microspheres are suitable for delivering octreotide compounds for all of their indications and uses. The “lyophilized pharmaceutical formulation” according to the disclosure can be administered intramuscularly, subcutaneously, or orally in the form of a suspension in a suitable liquid carrier. Accordingly, also provided by the disclosure is a method of treating a disease, disorder or condition in a warm blooded species (e.g., a mammal including a human patient) in need of such treatment. This method comprises use of the pharmaceutical formulation of the disclosure to administer an octreotide compound to the patient. While any suitable means of administration to a patient can be used within the context of the disclosure, typically the inventive method of treating a disease in a patient involves administering the pharmaceutical formulation to a patient via injection. By the term “injection,” it is meant that the composition is forcefully introduced into a target tissue of the patient. The composition can be administered to the patient by any suitable route, but is specifically administered to the patient intramuscularly or subcutaneously. When the inventive pharmaceutical formulation is administered by injecting, any suitable injection device can be used. Other routes of administration can be used to deliver the composition to the patient in accordance with the inventive method. Indeed, although more than one route can be used to administer the inventive formulation, a particular route can provide a more immediate and more effective reaction than another route.

According to yet another aspect of the disclosure, a pharmaceutical formulation and a method of producing it are provided. The pharmaceutical formulation utilizes a container, e.g., containing a single dose of microspheres containing an octreotide compound for treating a condition that is treatable by the sustained release of octreotide active agent from the microspheres and suspending agents. The amount of microspheres and suspending agents in the single dose is dependent upon the amount of active agent present in each container. Specifically, the single dose is selected to achieve the sustained release of the active agent over a period of from about 1 to about 180 days with the desired release profile.

The microspheres can be administered alone, or in appropriate combination with other active agents or drug therapies, as part of a pharmaceutical formulation. Such a pharmaceutical formulation may include the microspheres in combination with any standard physiologically and/or pharmaceutically acceptable carriers which are known in the art. The formulation compositions should be sterile and contain a therapeutically effective amount of the microsphere in a unit of weight or volume suitable for administration to a patient. The term “pharmaceutically-acceptable carrier” as used herein means one or more compatible solid or liquid fillers, diluents or encapsulating substances which are suitable for administration into a human or other mammal. The term “carrier” denotes an organic or inorganic ingredient, natural or synthetic, with which the active ingredient-containing microspheres are combined to facilitate the application. The components of the pharmaceutical formulation are capable of being co-mingled with the components of the present disclosure (e.g., the active agent, the biodegradable polymer), and with each other, in a manner such that there is no interaction that substantially impairs the desired pharmaceutical efficacy. Pharmaceutically acceptable carrier further means a non-toxic material that is compatible with a biological system such as a cell, cell culture, tissue, or organism. The characteristics of the carrier depend on the route of administration. Physiologically and pharmaceutically acceptable carriers include diluents, fillers, salts, buffers, stabilizers, desiccants, bulking agents, propellants, acidifying agents, coating agents, solubilizers, and other materials which are well known in the art. Carrier formulations suitable for oral, subcutaneous, intravenous, intramuscular, or other type of administrations also are well known, and can be found, e.g., in Remington's Pharmaceutical Sciences (Mack Publishing Co., Easton, Pa.), as well as in other sources. The “pharmaceutically-acceptable carrier” according to the disclosure can be bulking agents and wetting agents, for example, sodium carboxymethylcellulose and mannitol. The amount of sodium carboxymethylcellulose in the formulation ranges from 0.1% to 10%, even more specifically about 1.5% to about 5.0% by weight of the pharmaceutical formulation. The amount of mannitol in the formulation ranges from 10% to 50%, even more specifically about 18% to about 21% by weight of the pharmaceutical formulation.

Preparations for parenteral administration include but are not limited to sterile aqueous or non-aqueous solutions, suspensions, and emulsions. Examples of solvents include propylene glycol, polyethylene glycol, and vegetable oils such as olive oil, injectable organic esters such as ethyl oleate, and the like. Aqueous carriers include water, salts and buffer solutions such as saline and buffered media, alcoholic/aqueous solutions and emulsions or suspensions, as well as others. Parenteral vehicles include but are not limited to Normal Saline (0.9% sodium chloride), ½ Normal Saline (0.45% sodium chloride), 5% Dextrose in Water, Lactated Ringer's Solution, 5% Dextrose in ½ Normal Saline with 20 mEq KCl, 5% Dextrose in Lactated Ringer's Solution, 5% Dextrose in ⅓ Normal Saline, 5% dextrose in ½ Normal Saline, Normosol®-M in 5% Dextrose, Normosol®-R in 5% Dextrose, as well as others. Intravenous vehicles include fluid and nutrient replenishers, electrolyte replenishers (such as those based on Ringer's dextrose), and the like. Preservatives and other additives also optionally can be present such as, for example, antimicrobials, anti-oxidants, chelating agents, and inert gases and the like, so long as these additional ingredients do not deleteriously impact the advantageous properties of the microspheres. The “reconstitution solvent” according to the disclosure can be an aqueous carrier, specifically, water for injection. The amount of water for injection can be used for reconstitution and ranges from about 1 mL to about 5 mL, even more specifically about 2 mL to about 3 mL.

The “octreotide loaded microspheres” according to the disclosure generally have a spherical shape and range in size from about 0.1 microns to about 500 micrometers in diameter, even more specifically from about 1 to about 200 microns, depending upon the fabrication conditions. The octreotide content in the octreotide loaded microspheres ranges from 1% to 10% of weight of the microspheres, even more specifically 4% to 6%. The microspheres can be employed as a “delivery system” to release active agent from the interior of the microsphere (it can be released from the interior and exterior of the microspheres, e.g., a surface associated drug), when placed in an appropriate aqueous medium (e.g., such as in body fluids, in a physiologically acceptable buffer, or in any appropriate aqueous environment). As used herein, the term “sustained-release” refers to the release of an active agent from the microspheres of the disclosure over a defined or extended period of time in a continuous, discontinuous, linear or nonlinear manner. For example, release may be essentially biphasic, e.g., as where the release includes an initial release (a controlled or suppressed release, in which less than about 5% of active agent is released from the formulation in 1 hour, even more specifically less than about 1% in 1 hour at pH 7.4), followed by relatively continuous release of the active agent from the microsphere over time. Methods of measuring release are well known in the art (see, e.g., Hora et al., Pharm. Res. 7:1190-1194 (1990); Hora et al., Bio/Technology 8:755-758 (1990)). According to the disclosure sustained release can be continuous, relatively linear, and prolonged (i.e., as opposed to being short-lived).

The polymer is a biodegradable and biocompatible polymer, especially a polyester. Polyesters are particularly suited for the methods and compositions of the present disclosure because of their characteristically low human toxicity and virtually complete biodegradability. Such polyesters for use herein are polyglycolic (PGA) and polylactic (PLA) acids, and copolymers of glycolic acid and L-lactic acid (i.e., poly(D,L-lactide-co-glycolide) or PLGA). These polymers are available in a variety of molecular weights, and the appropriate molecular weight to provide the desired release rate for the octreotide active agent is readily determined by one of skill in the art. Thus, for instance, for PLA, a suitable molecular weight is on the order of from about 2000 to 250,000 daltons. For PLGA, suitable molecular weights generally range from about 10,000 to about 200,000 daltons, more specifically from about 15,000 to about 150,000 daltons, and most specifically from about 30,000 to about 60,000 daltons. If a polymer (i.e., a copolymer) such as PLGA is used to form the microspheres, a variety of lactic acid:glycolic acid ratios are applicable herein, and the ratio is largely a matter of choice, depending in part on the rate of degradation desired. For example, a 50:50 PLGA polymer, containing 50% D,L-lactide and 50% glycolide, is a fast resorbing polymer while 75:25 PLGA degrades more slowly, and 85:15 and 90:10, even more slowly, due to the increased lactide component. It is readily apparent that a suitable ratio of lactide:glycolide is easily determined by one of skill in the art based on the nature of the disorder to be treated. Moreover, mixtures of microspheres with varying lactide:glycolide ratios can be employed in the formulations of the disclosure to achieve the desired release kinetics.

More specifically, the biodegradable polymer is a copolymer of lactic acid and glycolic acid (PLGA) with unit proportions (molar ratio) ranging from 40:60 to 75:25, and especially with unit proportions ranging from 50:50 (i.e., a “PLGA 50:50 polymer”), or is a mixture or blend of separate polymers of lactic acid and glycolic acid, or PLGA polymer that provides an average molar ratio of lactide:glycolide of 50:50.

Even though the selection of particular monomer ratios of lactic acid to glycolic acid in the polymer can be readily modified by one of ordinary skill in the art as discussed above, the disclosure advantageously does not use sugar modified PLGA. That is, there are no sugar moieties such as glucose bonded to the polymer chain, as there are in the “star polymer” disclosed in U.S. Pat. No. 5,538,739 and used in Sandostatin LAR. The present disclosure does not employ the star polymer. The present disclosure, in forming the microspheres having an extended release substantially without an initial burst, includes method features in a particular O/W process which are at least in part due to using the conventional PLGA polymer without sugar moieties. These method features include, but are not limited to, adjusting concentration of polymer and/or acid in the dispersed phase as discussed in the examples below.

The term octreotide includes its analogues or derivatives thereof. The terms derivatives and analogues mean branched, straight chain or cyclic polypeptides in which at least one of the amino acids has been omitted or substituted by at least one other amino acid radical(s); and also include at least one functional group being substituted for at least one other functional group(s); and at least one group being substituted by at least one other isosteric group(s). In a broad sense, the terms mean all modified derivatives of octreotide that are biologically active and have a similar effect as unmodified octreotide.

The term “octreotide compound” means octreotide as a free base, salt or complex. Acid addition salts may be formed by inorganic or organic acids or polymeric acids. This includes octreotide acetate. Complexes might be formed by addition of octreotide and inorganic compounds.

In this disclosure a method for preparing microspheres for extended release of an octreotide compound with a low initial burst includes dissolving poly(D,L-lactide-co-glycolide) polymer in dichloromethane to form a polymer solution. The concentration of the polymer ranges from about 13% to about 15% of the polymer solution. Octreotide acetate is dissolved in a mixture of glacial acetic acid and methanol to form an octreotide solution. A concentration of the octreotide acetate ranges from about 9.2% to about 10.9% of the octreotide solution. A concentration of the glacial acetic acid ranges from about 5 to 10% and, in particular, from about 5.7% to about 6.7% of the octreotide solution. The polymer solution and the octreotide solution are mixed together to form a dispersed phase. A concentration of the polymer ranges from about 10 to 20% and, in particular, from about 12% to about 15% of the dispersed phase. A concentration of the octreotide compound ranges from about 1 to 5% and, in particular, from about 0.8% to about 1.0% of the dispersed phase. A concentration of the glacial acetic acid ranges from about 1 to 5% and, in particular, from about 0.4% to about 0.6% of the dispersed phase. Polyvinyl alcohol is dissolved in water at 0.35% to form a continuous phase. The dispersed phase is mixed with the continuous phase to form a microsphere suspension. The suspension is believed to be formed by nearly immediate emulsification of the dispersed phase in the continuous phase. Dichloromethane, acetic acid, methanol and polyvinyl alcohol are removed from the suspension. Residual organic solvents (dichloromethane and methanol) are removed from the microspheres by washing. These solvent removal steps occur by washing with room temperature water and warm water.

The water of the suspension is exchanged with a diluent solution, which comprises sodium carboxymethylcellulose and mannitol. A concentration of the octreotide acetate in the microsphere suspension is then adjusted. The microsphere suspension is filled into vials and then lyophilized. The vials are stoppered and sealed. The lyophilized octreotide suspension is a pharmaceutical formulation for injection.

As indicated above, one or more organic solvents are used, which can be pharmaceutically or pharmacologically acceptable. By “pharmaceutically acceptable” or “pharmacologically acceptable” is meant a material which is not biologically or otherwise undesirable, i.e., the material may be administered to a being or individual along with or as part of the microsphere formulations without causing any unnecessary undesirable biological effects or interacting in a deleterious manner with any of the components of the composition in which it is contained. The biodegradable polymer is dissolved to produce the polymer solution using an organic solvent that can be immiscible with water and is a volatile solvent. Examples of organic solvents that can be employed include halogenated hydrocarbons (e.g. dichloromethane, chloroform, chloroethane, trichloroethane, carbon tetrachloride, and the like), alkyl ethers having 3 or more carbon atoms (e.g. isopropylether), fatty acid alkyl (having 4 or more carbon atoms) esters (e.g. butyl acetate), aromatic hydrocarbons (e.g. benzene, toluene, xylene), as well as others. These solvents can be used alone or in combinations thereof. Specific halogenated hydrocarbons (e.g. dichloromethane, chloroform, chloroethane, trichloroethane, carbon tetrachloride, etc.) can be used, and in particular, the organic solvent is dichloromethane.

The polymer can be dissolved in a small amount of the organic solvent, reflective of its weight in the microsphere ultimately obtained. The concentration of polymer in the organic solvent is about 0.5% to 50% (w/w), specifically about 5% to 20%, more specifically 10% to 15%. To the mixture of glacial acetic acid and methanol, octreotide is dissolved to produce the octreotide solution. The organic solvents for the octreotide solution specifically include but are not limited to the solvents methanol, ethanol, dimethylacetamide (DMA), tetrahydrofuran (THF), dioxane, dimethylsulfoxide (DMSO), acetic acid, lactic acid, and dimethylformamide (DMF). In particular, the organic solvents are methanol and glacial acetic acid.

The concentration of octreotide in the solution is about 0.1% to 50%, in particular about 1% to 20%, more specifically 5% to 15%.

The homogeneous organic dispersed phase is emulsified into aqueous surfactant solution to form an oil-in-water (O/W) emulsion. The emulsification can be carried out by conventional dispersion techniques such as intermittent shaking, mixing by means of a mixer, colloid mill operation, mechanical homogenization, ultrasonication, and the like. Specifically, the emulsification is done in an aqueous dispersed phase containing a surfactant, especially polyvinyl alcohol (PVA) in water. Examples of other emulsifiers that optionally can be employed include anionic surfactants (e.g., sodium oleate, sodium stearate, sodium lauryl sulfate), non-ionic surfactants (e.g., polyoxy-ethylene-sorbitan fatty acid esters [Tween 80 or Tween 60, e.g., from Atlas Powder], polyoxyethylene-castor oil derivatives [HCO-60 or HCO-50 from Nikko Chemicals], or others), polyvinylpyrrolidone, carboxymethylcellulose, lecithin, gelatin, and hyaluronic acid. The surfactant amount (e.g., the PVA amount) ranges from about 0.01 to about 10% (w/v), more specifically 0.1% to 1%.

Removal of the organic solvent from the produced microspheres can be carried out by conventional methods. Examples of the removal method of the organic solvent include but are not limited to spray drying, phase separation, and in-water drying. For instance, the removal of the organic solvent can be carried out by evaporating the organic solvent by stirring with a propeller-type stirrer, magnetic stirrer, or the like, optionally under atmospheric pressure, or gradually reducing pressure while controlling the degree of vacuum, e.g., by using a rotary evaporator. These methods are routine.

The organic solvents can be removed by extraction and washing with cold and warm water, which further solidifies the microspheres. This is done by increasing the temperature to from about 20° C. to about 36° C., and stirring for from about 30 minutes to about 60 minutes. In particular, this is done by increasing the temperature to about 34° C. to 38° C., and stirring for about 30 minutes to 60 minutes.

A specific process for making the microspheres is as follows. A dispersed phase is made by dissolving polymer and octreotide acetate in a solvent mixture. The PLGA polymer is dissolved in a suitable solvent (e.g., dichloromethane). The octreotide acetate is dissolved in acid (e.g., acetic acid) and a suitable solvent (e.g., methanol). The solvent for the drug is a nonsolvent for the polymer and the solvent for the polymer is a nonsolvent for the drug. The polymer and octreotide solutions are true, filterable solutions. It will be apparent that the octreotide compound, polymer, their solvents and the acid could be added separately or all together at the same time. The selection of particular solvents and continuous phases can be varied depending upon the intended product characteristics.

The continuous phase is charged in a vessel equipped with temperature control. This disclosure can use a Silverson homogenizer (Model L4RT from Silverson Machines) equipped with a standard emulsor screen (for batch processing) or a specially designed in-line Silverson mixer (for continuous processing) as disclosed in U.S. Pat. No. 5,945,126, which is incorporated herein by reference in its entirety. The Silverson homogenizer is charged with the continuous phase. In batch processing the dispersed phase is drawn into a syringe and added to the continuous phase while mixing, just below the mixing head using a long (12″) syringe needle bent appropriately to reach the position below the mixing head. The continuous process adds to the modified Silverson homogenizer the dispersed phase and the continuous phase at certain flow rates specified in the U.S. Pat. No. 5,945,126 patent. The U.S. Pat. No. 5,945,126 patent may be referred to for various aspects of the continuous microsphere process.

The dispersed phase is dispersed or emulsified in the continuous phase to form droplets or inclusions of the dispersed phase in the continuous phase. The terms emulsified or dispersed are intended in their broadest sense as meaning discrete regions of dispersed phase interspersed within the continuous phase. The noted inclusions will occur as generally spherical droplets, but may in some instances be irregular inclusions due to particular emulsification conditions. Any suitable medium in which the dispersed phase will form droplets or inclusions may be used as a continuous phase, with those that provide a maximum solvent sink for the dispersed phase solvent being especially desirable. The continuous phase might also contain surfactant, stabilizers, salts or other additives that modify or affect the emulsification process.

The particular continuous phase is water. The aqueous continuous phase will typically contain a surfactant or emulsifier, such as polyvinyl alcohol, in an amount of from about 0.1% to about 5%. Examples of other emulsifiers that optionally can be employed include anionic surfactants (e.g., sodium oleate, sodium stearate, sodium lauryl sulfate), non-ionic surfactants (e.g., polyoxy-ethylene-sorbitan fatty acid esters [Tween 80 or Tween 60, e.g., from Atlas Powder], polyoxyethylene-castor oil derivatives [HCO-60 or HCO-50 from Nikko Chemicals], or others), polyvinylpyrrolidone, carboxymethylcellulose, lecithin, gelatin, and hyaluronic acid. These emulsifiers (and/or surfactants) can be used independently or in combination.

After the dispersed phase addition is complete, the microsphere suspension is mixed at a lower speed for solvent removal. This could be carried out in a solvent removal vessel (e.g., an Applikon bioreactor). Solvent removal is achieved by exchanging the continuous phase with room temperature water, followed by hot water (30-40° C.), followed by room temperature water. The room temperature water removes external phase solvent; the hot water removes internal solvent from the microspheres and then the microspheres are returned to room temperature water for further processing. An optional air sweep is used at the surface of the stirring suspension to remove the headspace solvent during the solvent removal process. The microspheres are filtered on a Durapore membrane filter using an Amicon stir cell assembly. The microspheres are washed with water to remove residual stabilizer (e.g., PVA). They are then dried at low temperature (<25° C.) under a vacuum.

The solidified microspheres containing octreotide are uniformly suspended in a diluent solution that contains sodium carboxymethylcellulose and mannitol. The concentration of mannitol in the microsphere suspension ranges from about 10 mg/g to 100 mg/g, specifically 30 mg/g to 60 mg/g. The concentration of sodium carboxymethylcellulose in the microsphere suspension ranges from about 1 mg/g to 20 mg/g, specifically 2 mg/g to 15 mg/g. The suspension of octreotide-loaded microspheres are filled into a container, e.g. glass vials, and lyophilized.

The suspension filled vials can be lyophilized using a lyophilization method. For example, the vials are chilled to a temperature from about −10° C. to about +5° C., wherein the temperature is maintained for at least about 20 minutes to about 3 hours. The vials are frozen to a temperature of from about −10° C. to about −70° C. to produce a frozen mixture. The temperature is maintained for at least about 30 minutes to about 20 hours. The frozen mixture is subjected to a primary drying stage, which comprises applying a vacuum to reduce the pressure by an amount effective to remove aqueous solvent from the frozen mixture and, while applying the vacuum, changing (e.g., raising or lowering) the temperature of the frozen mixture to a primary drying temperature. The primary drying temperature is from about −30° C. to about 20° C. The primary drying temperature is maintained for at least about 15 hours to about 50 hours, to produce a first intermediate. The first intermediate is subjected to a secondary drying stage, which comprises applying a vacuum to reduce the pressure by an amount effective to remove aqueous solvent from the first intermediate and, while applying the vacuum, changing (e.g., raising or lowering) the temperature of the first intermediate to a first secondary drying temperature. The first secondary drying temperature is from about 0° C. to about 45° C. The first secondary drying temperature is maintained for at least about 5 hours to about 30 hours. The temperature of the first intermediate is changed (e.g., raised or lowered) to a second secondary drying temperature, wherein the second secondary drying temperature is from about 0 to about 60° C. The second secondary drying temperature is maintained for at least about 5 hours to about 30 hours, to produce the pharmaceutical formulation.

The lyophilized formulation of the present disclosure is a white to slightly yellow lyophilized cake or powder of octreotide containing PLGA microspheres, sodium carboxymethyl cellulose and mannitol. The lyophilized octreotide of the present disclosure can have a purity of about 90% or greater (i.e., contains about 10% or less of total impurities based on the total weight of octreotide), and more specifically has a purity of about 95% or greater. Purity can be determined by high performance liquid chromatography assay (e.g., allowing separation of pure lyophilized octreotide from impurities, and quantitation of the relative amounts by the determination of the peak area of pure octreotide as compared to total peak area), or by a similar method, and excludes moisture of the octreotide acetate, and acetate itself.

The lyophilized octreotide sustained release formulation can comprise any suitable amount of octreotide, but ideally comprises a therapeutically effective amount of octreotide. A “therapeutically effective amount” means an amount sufficient to show a meaningful benefit in an individual, e.g., promoting at least one aspect of treatment, healing or prevention of other relevant medical condition(s) such as that associated with acromegaley and cancer syndromes. Therapeutically effective amounts may vary depending upon the biological effect desired in the individual, condition to be treated, and the individual. In this regard, the lyophilized octreotide can be present in the sustained formulation in an amount from about 5 mg to about 50 mg (e.g., about 5 mg, about 10 mg, about 20 mg, about 30 mg, or about 50 mg). More specifically, the lyophilized octreotide is present in an amount from about 10 mg to about 30 mg (e.g., about 10 mg, about 20 mg, or about 30 mg).

The lyophilized octreotide microsphere formulation has low moisture content. The moisture content of the inventive lyophilized octreotide microsphere formulation is the result of residual water that remains in the formulation after the lyophilization process. The moisture content can be the product of any suitable solvent that is used in the method of producing the lyophilized octreotide microsphere formulation described herein. The lyophilized octreotide microsphere formulation can have a moisture content of less than from about 0.01 wt % to about 10 wt %, where the wt % is the % water relative to the dry weight of the lyophilized octreotide microsphere formulation.

The inventive lyophilized octreotide microsphere formulation according to the disclosure can be contained within a sealed container. Each octreotide formulation can be contained within a container that is sealed aseptically. The container can be provided with an opening and a means for aseptically sealing the opening, e.g., such that the sealed container is fluidly sealed or the sealed opening is substantially impermeable to atmospheric gasses, moisture, pathogenic microorganisms, or the like. The container can be constructed of any suitable material such as, for example, glass, polypropylene, Daikyo Resin CZ (sold by Daikyo Gomu Seiko, Ltd.), polyethylene terephthalate, and the like. In particular, the container is constructed of glass. Suitable glass containers include, but are not limited to, glass vials.

A suitable means for sealing the container can include, for example, a stopper, a cap, a lid, a closure, a covering which fluidly seals the container, or the like. Examples of suitable closures include closures that are suitable for medical vials, such as those described in U.S. Pat. No. 4,671,331, and references cited therein. The means for sealing the container are not limited to separate closures or closure devices, but also includes self-sealing containers and containers which are manufactured and sealed during filling operations. The means for aseptically sealing the container can include a stopper such as, for example, a stopper that is configured to fluidly seal the opening.

An outer seal is provided which covers and entirely surrounds the stopper. The outer seal can be constructed of any suitable material. When an outer seal is used, it is fitted with a lid that can be easily manually removed to provide access to the stopper. Such seals include an outer rim made of a suitable material, such as aluminum, that entirely surrounds the lateral edge of the stopper and further include a lid (typically polypropylene or other suitable material) that entirely covers the upper surface of the stopper. The polypropylene lid can be “flipped” off e.g., by exerting upward pressure with a finger or thumb, to provide access to the stopper, e.g., so that it can be punctured with a hypodermic needle to deliver an aqueous vehicle for constitution (see, e.g., U.S. Pat. No. 6,136,814).

The disclosure further provides a solution prepared by suspending the inventive lyophilized octreotide microsphere formulation in an aqueous vehicle. The aqueous vehicle can be a sterile aqueous vehicle that is normally used as liquid vehicle for injection. Suitable aqueous vehicles include, for example, sterile water (e.g., Sterile Water for Injection, USP), sodium chloride solutions (e.g., 0.9% Sodium Chloride for Injection, USP), dextrose solutions (e.g., 10% Dextrose for Injection), sodium chloride/dextrose mixtures (e.g., 5% Dextrose and 0.225% Sodium Chloride for Injection, 5% Dextrose and 0.45% Sodium Chloride for Injection), Lactated Ringer's for Injection, and mixtures thereof.

The inventive lyophilized octreotide microsphere formulation can be suspended in any suitable volume of the aqueous vehicle. Specifically, the lyophilized octreotide microspheres are suspended in about 10 mL or less (e.g., about 10 mL, about 8 mL, about 6 mL, about 4 mL, or about 1 mL) of the aqueous vehicle. The lyophilized octreotide can be suspended in about 1 mL to about 5 mL of the aqueous vehicle. More specifically, the lyophilized octreotide acetate microspheres are suspended in about 2 mL to about 3 mL of the aqueous vehicle.

The disclosure will now be described by reference to the following examples, which should not be used to limit the invention as described in the appended claims.

### EXAMPLE 1

Octreotide Loaded PLGA Microshperes with High Initial Burst Release

These octreotide PLGA microspheres were manufactured with about 5% (w/w) glacial acetic acid in the dispersed phase. The microspheres showed about 2.4% initial release within 5 minutes at pH 7 and about 2.8% initial release within 15 minutes at pH 4. Briefly, the microspheres were manufactured as follows. 9.36 g of poly(D,L-lactide-co-glycolide) (PLGA, lactide:glycolide=50:50, inherent viscosity=0.60 g/dL) was dissolved in 63.66 g of dichloromethane to prepare the polymer solution. Separately, 0.76 g of octreotide acetate was dissolved in a mixture of 0.40 g glacial acetic acid and 5.99 g methanol to prepare the octreotide solution. The octreotide solution was added to the polymer solution, and then mixed to prepare a clear and slightly yellow dispersed phase (DP). Separately, 0.35% polyvinyl alcohol was dissolved in purified water and filtered through 0.22 micron PVDF membrane filter. This aqueous solution served as the continuous phase (CP). The DP and CP were pumped simultaneously at 40 ml/min and 2000 mL/min, respectively, into the Silverson in-line mixer, which was mixed at 4000 rpm. The DP was instantly emulsified; solidified octreotide loaded PLGA microspheres in the Silverson mixer were discharged to a 50-L stainless steel tank where the microsphere suspension was diluted with room temperature water at 4000 mL/min. The volume of the microsphere suspension was about 12 L. The collected microspheres in the tank were transferred to a 3-L Applikon glass vessel where the microsphere suspension was recirculated through the hollow fiber filter while removing the filtrate through the permeate port. The microsphere suspension was concentrated to 1.5 L in the vessel and washed using ambient temperature water. The organic solvents were removed from the microsphere suspension by washing with 34-37° C. water. After the washing and solvent removal, the microspheres were collected using a 5 micron filter and freeze dried. The microspheres were determined to contain about 4.8% octreotide (as the free base). The average particle size of the microspheres was about 59 micron (<50% cumulative volume fraction, CVF).

### EXAMPLE 2

Octreotide Loaded PLGA Microspheres with Low Initial Burst Release

These octreotide PLGA microspheres were manufactured with about 0.5% (w/w) glacial acetic acid in the dispersed phase. The microspheres showed about 0.14% initial release within 5 minutes at pH 7 and about 0.24% initial release within 15 minutes at pH 4. Based on this initial release, it is expected that the initial release will be less than 1% of a total amount of octreotide acetate at 37° C. and a pH of 7.4. Briefly, the microspheres were manufactured as follows. 9.34 g of poly(D,L-lactide-co-glycolide) (PLGA, lactide:glycolide=50:50, inherent viscosity=0.60 g/dL) was dissolved in 60.64 g of dichloromethane to prepare the polymer solution. Separately, 0.77 g of octreotide acetate was dissolved in a mixture of 0.4 g glacial acetic acid and 6.01 g methanol to prepare the octreotide solution. The octreotide solution was added to the polymer solution, and then mixed to prepare a clear and slightly yellow dispersed phase (DP). The concentration of the polymer in the DP was about 11.7%. Separately, 0.35% polyvinyl alcohol was dissolved in purified water and filtered through a 0.22 micron PVDF membrane filter. This aqueous solution served as the continuous phase (CP). The DP and CP were pumped simultaneously at 40 ml/min and 2000 mL/min, respectively, into the Silverson in-line mixer, which was mixed at 4000 rpm. The DP was instantly emulsified; solidified octreotide loaded PLGA microspheres in the Silverson mixer were discharged to a 50-L stainless steel tank where the microsphere suspension was diluted with room temperature water at 4000 mL/min. The volume of the microsphere suspension was about 12 L. The collected microspheres in the tank were transferred to a 3-L Applikon glass vessel where the microsphere suspension was recirculated through the hollow fiber filter while removing the filtrate through the permeate port. The microsphere suspension was concentrated to 1.5 L in the vessel and washed using ambient temperature water. The organic solvents were removed from the microsphere suspension by washing with 34-37° C. water. After the washing and solvent removal, the microsphere were collected using a 5 micron filter and freeze dried. The microspheres were determined to contain about 4.5% octreotide (as the free base). The average particle size of the microspheres was about 58 micron (<50% CVF).

### EXAMPLE 3

Octreotide Loaded PLGA Microspheres with Very Low Initial Burst Release

These octreotide PLGA microspheres were manufactured with about 0.5% (w/w) glacial acetic acid and 14% polymer in the dispersed phase. The microspheres showed about 0.03% initial release within 15 minutes at pH 4. Based on this initial release, it is expected that the initial release will be less than 1% of a total amount of octreotide acetate at 37° C. and a pH of 7.4. Briefly, the microspheres were manufactured as follows. 9.34 g of poly(D,L-lactide-co-glycolide) (PLGA, lactide:glycolide=50:50, inherent viscosity=0.45 g/dL) was dissolved in 51.79 g of dichloromethane to prepare the polymer solution. Separately, 0.76 g of octreotide acetate was dissolved in a mixture of 0.40 g glacial acetic acid and 4.91 g methanol to prepare the octreotide solution. The octreotide solution was added to the polymer solution, and then mixed to prepare a clear and slightly yellow dispersed phase (DP). Separately, 0.35% polyvinyl alcohol was dissolved in purified water and filtered through 0.22 micron polyvinylidenefluoride (PVDF) membrane filter. This aqueous solution served as the continuous phase (CP). The DP and CP were pumped simultaneously at 40 ml/min and 4000 mL/min, respectively, into the Silverson in-line mixer, which was mixed at 5000 rpm. The DP was instantly emulsified; solidified octreotide loaded PLGA microspheres in the Silverson mixer were discharged to a 50-L stainless steel tank where the microsphere suspension was diluted with room temperature water at 2000 mL/min. The collected microspheres in the tank were transferred to a 3-L Applikon glass vessel where the microsphere suspension was recirculated through the hollow fiber filter while removing the filtrate through the permeate port. The microsphere suspension was concentrated to 1.5 L in the vessel and washed using ambient temperature water. The organic solvents were removed from the microsphere suspension by washing with 34-38° C. water. After the washing and solvent removal, the microspheres were collected using a 5 micron filter and freeze dried. The microspheres were determined to contain about 4.8% octreotide (as the free base). The average particle size of the microspheres was about 36 micron (<50% CVF).

### EXAMPLE 4

Effect of Acetic Acid Concentration in Dispersed Phase on Initial Burst

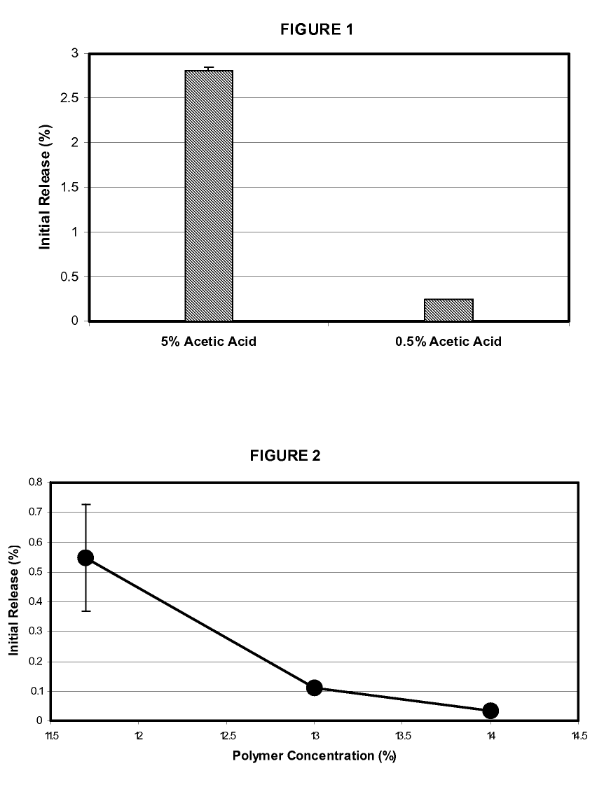

Two different lots of octreotide PLGA microspheres were prepared with different glacial acetic acid concentrations in the dispersed phase; 5% and 0.5%. The microspheres prepared using 5% acetic acid were produced as described in Example 1, while the microspheres prepared using 0.5% acetic acid were prepared by otherwise identical process parameters. The initial release within 15 minutes in pH 4 buffer was determined for the lots. The initial release decreased from 2.8% to 0.24% with a reduced amount of glacial acetic acid in the dispersed phase as seen in FIG. 1.

### EXAMPLE 5

Effect of PLGA Polymer Concentration in Dispersed Phase on Initial Burst

Three different lots of octreotide PLGA microspheres were prepared with different PLGA concentrations in the dispersed phase; 11.7%, 13.0% and 14%. The microspheres having 14% polymer were prepared as described in Example 3, while the microspheres having 11.7% and 13.0% polymer were prepared by otherwise identical process parameters. The initial release within 15 minutes in pH 4 buffer was determined for the lots. As seen in FIG. 2, the initial release decreased from 0.55% (polymer concentration=11.7%) to 0.034% (polymer concentration=14.0%) with increased concentration of polymer in the dispersed phase.

### EXAMPLE 6

Preparation of Lyophilized Octreotide Formulation

This lyophilized octreotide PLGA microsphere product was manufactured for the target octreotide content in the single dose vial of 30 mg/vial. This was prepared with about 0.5% (w/w) glacial acetic acid and 11.7% polymer in the dispersed phase. The lot size for the microspheres was 100 g. Briefly, the microspheres were manufactured as the follows. 93.54 g of poly(D,L-lactide-co-glycolide) (PLGA, lactide:glycolide=50:50, inherent viscosity=0.45 g/dL) was dissolved in 636.23 g of dichloromethane to prepare the polymer solution. Separately, 8.48 g of octreotide acetate was dissolved in a mixture of 4.00 g glacial acetic acid and 60.07 g methanol to prepare the octreotide solution. The octreotide solution was added to the polymer solution, and then mixed to prepare a clear and slightly yellow dispersed phase (DP). The DP was filtered through 0.22 micron PTFE filter membrane. Separately, 0.35% polyvinyl alcohol was dissolved in purified water and filtered through a 0.22 micron PVDF membrane filter. This aqueous solution served as the continuous phase (CP). The DP and CP were pumped simultaneously at 40 ml/min and 2100 mL/min, respectively, into the Silverson in-line mixer, which was mixed at 4000 rpm. The DP was instantly emulsified; solidified octreotide loaded PLGA microspheres were formed in the Silverson mixer and then discharged to a 100-L stainless steel tank where the microsphere suspension was diluted with room temperature water at 4000 mL/min. The collected microspheres in the tank were transferred to a 3-L Applikon glass vessel where the microsphere suspension was recirculated through the hollow fiber filter while removing the filtrate through the permeate port. The microspheres suspension was concentrated to 1.5L in the vessel and washed using ambient temperature water, which removed external phase solvent. The internal organic solvents were removed from the microspheres of the suspension by washing with 34-38° C. water; then the suspension was returned to room temperature.

After the washing and solvent removal, the microsphere suspension was suspended in the diluent solution which contained 2.8 mg/g sodium carboxymethyl cellulose and 30.6 mg/g mannitol. The octreotide concentration in the suspension was 8.03 mg/g as the octreotide free base. The suspension was diluted to the target octreotide concentration of 6.67 mg/g using the diluent solution. The concentration was then determined to be 6.87 mg/g. The final weight of the suspension was 617 g.

While stirring the suspension, 4.5 g suspension was filled into 5-cc glass vials. A total of 124 vials were filled and half-stoppered using the West 4432 lyophilization stoppers. The vials were loaded in the Virtis lab lyophilizer and lyophilized as disclosed herein for about 31 hours. The lyophilized vials were fully stoppered under a slight vacuum and unloaded from the lyophilizer, and sealed using flip-off aluminum seals. The vials were determined to contain an average of 30.57 mg octreotide free base/vial. The average particle size was about 52 micron (<50% CVF). The lot showed about 0.30% initial release within 5 minutes in pH 7.4 and about 0.42% initial release within 15 minutes at pH 4. Based on this initial release, it is expected that the initial release will be less than 1% of a total amount of octreotide acetate within 1 hour at 37° C. and a pH of 7.4. The moisture content was 0.34% and the total impurities were about 3.3%. The residual dichloromethane was determined to be about 1646 ppm.

### EXAMPLE 7

In Vivo Release in Rats

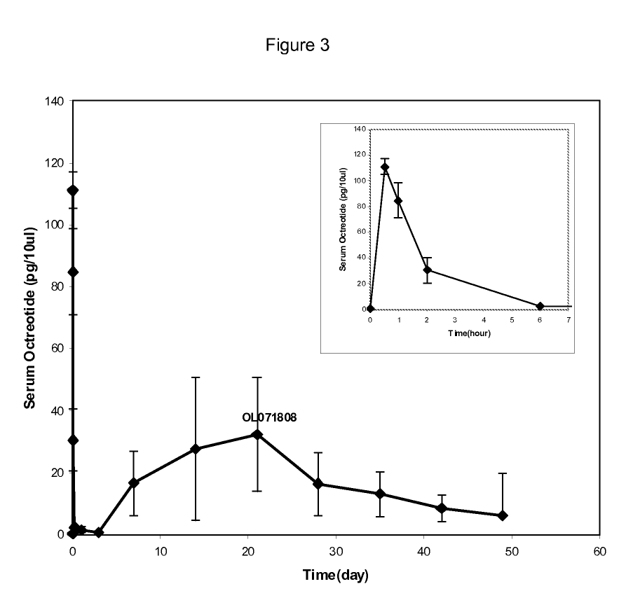

The microspheres prepared in Example 6 were used. After a single intramuscular injection of the lyophilized octreotide formulation in rats (target dose=3 mg octreotide/rat), the octreotide concentration in serum was monitored at predetermined time points using a radioimmuno assay method. A total of 8 rats were used for this study. The actual dose was about 3.8 mg/rat. Referring to FIG. 3, the serum octreotide concentration reached an initial peak of about 110 pg/10 uL within 30 minutes after the administration, declining over 3 days to 0.8 pg/10 uL, then slowly increasing to about 28 pg/10 uL in 3 weeks reaching to about 30 pg/10 uL. The serum octreotide concentration declined gradually after 21 days and reached about 6 pg/10 uL at 49 days.

### EXAMPLE 8

Suspendability and Syringeability

The suspendability of octreotide loaded PLGA microsphere formulations was determined by the reconstitution time after adding resuspending medium. Faster reconstitution time represents better suspendability. The syringeability was determined using the resuspended octreotide microsphere suspension and different bore size needles. The suspension being syringeable through smaller bore needles represents better syringeability.

Three different samples of microspheres prepared as described herein were used in this test. The first sample was octreotide microspheres lyophilized with diluent composition (sodium carboxymethylcellulose and mannitol) in a 5-cc vial reconstituted with 2.5 mL water for injection. The second sample was a physical mixture of octreotide microspheres and lyophilized diluent composition that had been lyophilized separately in a 5-cc vial followed by reconstitution with 2.5 mL water for injection. The last sample was octreotide microspheres mixed with 2.5 mL liquid diluent that contained carboxymethylcellulose and mannitol. The time for a complete resuspension for microspheres was measured as the reconstitution time. The suspension was withdrawn using a 3-cc syringe equipped with different bore size needles and expelled to test the syringeability. Any blockage and clogging during the withdrawal and injection was regarded as a failure of syringeability. The following table summarizes the test results for the reconstitution time and syringeability.

TABLE 1
Reconstitution time and syringeability

| | | Syringebility | Syringebility | Syringebility |
|---|---|---|---|---|
| | | through 20G | through 22G | through 23G |
| | | 1.5″ needle | 1.5″ needle | 1.5″ needle |
| | Reconstitution | Inner diameter = | Inner diameter = | Inner diameter = |
| Sample | Time | 0.584 mm | 0.394 mm | 0.318 mm |
| Octreotide | 1 minute 10 | Syringeable | Syringeable | Syringeable |
| microspheres | seconds | | | |
| lyophilized with | | | | |
| diluent composition | | | | |
| Octreotide | 2 minutes 52 | Syringeable | Syringeable | Not Syringeable |
| microspheres with | seconds | | | |
| separately | | | | |
| lyophilized diluent | | | | |
| composition | | | | |
| Octreotide | 2 minutes 9 | Syringeable | Syringeable | Not Syringeable |
| microspheres with | seconds | | | |
| liquid diluent | | | | |
| composition | | | | |

Many modifications and variations of the invention will be apparent to those of ordinary skill in the art in light of the foregoing disclosure. Therefore, it is to be understood that, within the scope of the appended claims, the invention can be practiced otherwise than has been specifically shown and described.

What is claimed is:

1. Microspheres for sustained release of an octreotide compound with a low initial burst, comprising a poly(D,L-lactide-co-glycolide) polymer matrix and an octreotide compound dispersed in said polymer matrix, wherein said microspheres release less than 1% of a total amount of said octreotide compound within 1 hour at 37° C. and pH 7.4.

2. The microspheres of claim 1 containing less than 2000 ppm residual solvents.

3. The microspheres of claim 1 wherein said polymer has a molar ratio of lactide to glycolide ranging from 40:60 to 75:25.

4. The microspheres of claim 1 wherein said octreotide compound is selected from the group consisting of a free base, an acid addition salt and a complex of octreotide.

5. The microspheres of claim 1 wherein said octreotide compound is octreotide acetate.

6. A lyophilized pharmaceutical formulation comprising said microspheres of claim 1, sodium carboxymethylcellulose and mannitol.

7. The formulation of claim 6 wherein said octreotide compound is present in an amount of about 3% to about 6% based on a weight of the formulation.

8. The formulation of claim 6 wherein said polymer is present in an amount of about 70.0% to about 75.5% by weight of the formulation.

9. The formulation of claim 6 wherein said microspheres are present in the formulation in an amount of about 200 mg to about 600 mg.

10. The formulation of claim 6 wherein said sodium carboxymethyl cellulose is present in an amount of from about 1.5% to about 5.0% by weight of the formulation.

11. The formulation of claim 6 wherein said mannitol is present in an amount of from about 18% to about 21% by weight of the formulation.

12. The formulation of claim 6 which is one of an intramuscular or subcutaneous injectable formulation suitable for a mammal in need of said octreotide compound.

13. The formulation of claim 6 which is reconstituted with about 2 mL to about 3 mL water for injection.

14. The formulation of claim 12 which is reconstituted with water for injection and is injectable through a needle that has an inner diameter of 0.584 mm or smaller.

15. A process for preparing microspheres for extended release of an octreotide compound with a low initial burst comprising:
a) preparing a dispersed phase by combining poly(D,L-lactide-co-glycolide) polymer, dichloromethane, said octreotide compound, methanol, and acetic acid; wherein a concentration of said polymer ranges from about 10% to about 20% of said dispersed phase, a concentration of said octreotide compound ranges from about 0.1% to about 5.0% of said dispersed phase and a concentration of said acetic acid ranges from about 0.1% to about 5.0% of said dispersed phase;
b) dissolving polyvinyl alcohol in water to form a continuous phase;
c) mixing said dispersed phase in said continuous phase to form a microsphere suspension;
d) removing said dichloromethane, said acetic acid, said methanol and said polyvinyl alcohol from said microsphere suspension; and
e) removing residual dichloromethane and methanol from said microspheres by washing.

16. The method of claim 15 wherein a concentration of said polymer ranges from about 12% to about 15% of said dispersed phase, a concentration of said octreotide compound ranges from about 0.8% to about 1.0% of said dispersed phase and a concentration of said acetic acid ranges from about 0.4% to about 0.6% of said dispersed phase.

17. The method of claim 15 comprising f) adding a diluent to said microspheres after step e), said diluent comprising sodium carboxymethylcellulose and mannitol.

18. The method of claim 17 comprising g) adjusting a concentration of said octreotide compound in a microsphere suspension resulting from said step f).

19. The method of claim 18 comprising h) filling a suspension of said microspheres having said adjusted concentration of said octreotide compound into vials and lyophilizing the suspension in the filled vials.

20. The process according to claim 19 wherein a product of said lyophilization is a pharmaceutical formulation for injection.

21. A process for preparing microspheres for extended release of an octreotide compound with a controlled initial burst comprising
a) preparing a dispersed phase by combining poly(D,L-lactide-co-glycolide) polymer, a first solvent for said polymer, said octreotide compound, a second solvent for said octreotide compound and an acid compound;
b) mixing said dispersed phase in an aqueous continuous phase to form a microsphere suspension;
c) removing said first solvent, said acid compound, and said second solvent from said microsphere suspension;
d) removing residual said first and second solvents from said microspheres by washing;
e) measuring initial burst of said octreotide compound from said microspheres;
f) raising or lowering said initial burst to a desired level by adjusting a concentration of at least one of said polymer or said acid compound in said dispersed phase; and
g) repeating said steps a)-e) using said adjusted concentration of said polymer or said acid compound.

22. The method of claim 21 wherein said initial burst is lowered by increasing said concentration of said polymer in said dispersed phase as said adjusted concentration.

23. The method of claim 21 wherein said acid compound is acetic acid and said initial burst is lowered by decreasing a concentration of said acetic acid in said dispersed phase as said adjusted concentration.

24. The method of claim 21 wherein said first solvent is dichloromethane, said octreotide compound is octreotide acetate, said second solvent is methanol, said acid compound is acetic acid and said continuous phase includes polyvinyl alcohol.
